(19) United States
(12) Patent Application Publication
(10) Pub. No.: US 2022/0023580 A1
(43) Pub. Date: Jan. 27, 2022

(54) METHOD OF DELIVERING AN EXTRACT

(71) Applicant: John Sweeney, Oakland, CA (US)

(72) Inventor: John Sweeney, Oakland, CA (US)

(21) Appl. No.: 17/380,395

(22) Filed: Jul. 20, 2021

Related U.S. Application Data
(60) Provisional application No. 63/055,667, filed on Jul. 23, 2020.

(51) Int. Cl. A61M 16/14; D06M 13/00; A61K 36/185; A61K 31/352; A61K 31/05; A61M 16/06

(52) U.S. Cl. CPC A61M 16/14; D06M 13/005; A61K 36/185; A61M 2209/04; A61K 31/05; A61M 16/06; A61K 31/352

(57) ABSTRACT

A method of delivering liquid or solid extracts to a person is described. The method includes providing the extract to an inner surface of a face covering, such as a face mask, gator, or bandana. In certain embodiments, a reservoir or pad is provided to the inner surface to retain the extract. Once the person placed the face covering on their face, the extract vaporizes, providing a continuous flow of the extract to the person. The extract can be an extract of cannabis or an extract of hemp which contains THC or CBD, an extract of tobacco, an aromatherapy oil, or a pharmaceutical.

### CROSS-REFERENCE TO RELATED APPLICATIONS

This application claims the benefit of U.S. Provisional Application No. 63/055,667, filed Jul. 23, 2020, the contents of which are hereby incorporated by reference in its entirety.

### BACKGROUND OF THE INVENTION

### Field of the Invention

The present invention generally relates to methods for delivering extracts, and more particularly to methods for delivering extracts using a face covering or face mask.

### Discussion of the Background

Extracts are used by consumers to deliver active or desirable ingredients found, for example, in certain plants. Extracts are typically obtained by solvent extraction and are stored as a liquid or a solid. Extracts may be consumed directly, such as sublingually, or by inhalation, in which the extract is heated, vaporized, atomized, or otherwise converted into a form which can be inhaled.

The inhalation of extracts requires a delivery system, such as a vaporizer or a pressurized spray generator. While these delivery systems are effective, they have several problems, such as requiring that the consumer operate the delivery system for each dose of extract.

Thus, there is a need in the art for a method that permits for a more convenient way of delivering extracts. Such a method and apparatus should be easy to use and cost effective.

### BRIEF SUMMARY OF THE INVENTION

The disadvantages of prior art extract delivery systems are overcome by dispensing an extract using a face covering.

In certain embodiments, an extract is provided to a face covering, and a wearer then places the face covering on their face to cover the mouth and nose.

These features together with the various ancillary provisions and features which will become apparent to those skilled in the art from the following detailed description, are attained by the method of the present invention, preferred embodiments thereof being shown with reference to the accompanying drawings, by way of example only, wherein:

### BRIEF DESCRIPTION OF THE SEVERAL VIEWS OF THE DRAWING

FIG. 1 illustrates a prior art face covering;

FIG. 2 illustrates a first embodiment of a method of applying an extract to a face covering;

FIG. 3 illustrates a second embodiment of a method of applying an extract to a face covering;

FIG. 4 illustrates a third embodiment of a method of applying an extract to a face covering;

FIG. 5 illustrates a fourth embodiment of a method of applying an extract to a face covering; and

FIG. 6 illustrates next step of the first through fourth embodiments, where the face covering, and extract are placed on the face of wearer.

Reference symbols are used in the Figures to indicate certain components, aspects or features shown therein, with reference symbols common to more than one Figure indicating like components, aspects or features shown therein.

### DETAILED DESCRIPTION OF THE INVENTION

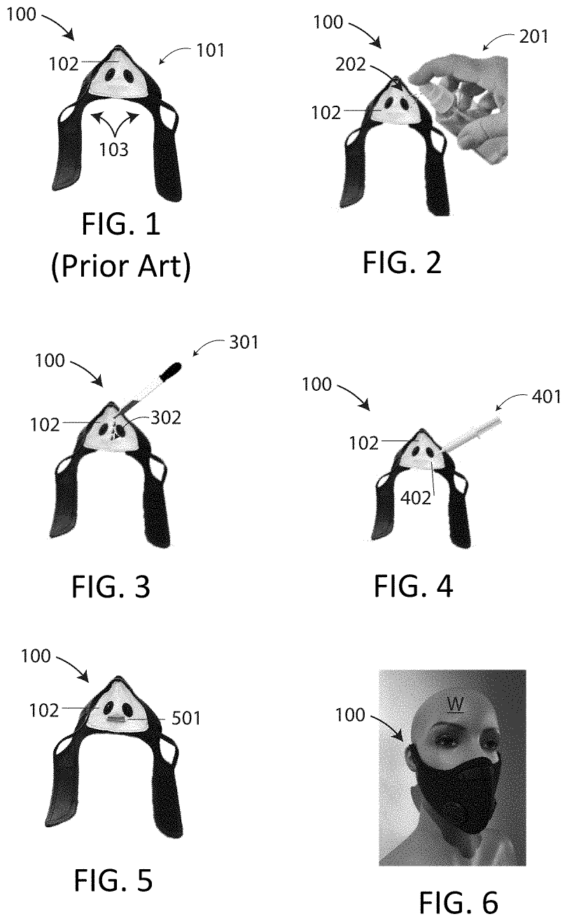

FIG. 1 illustrates a prior art face covering 100 which may be used, without limitation, in the methods described herein. The term “face covering,” as used herein, generically refers to an article or piece of clothing placeable on the face to cover least the mouth and nose of the wearer, and includes, without limitation, cloth or paper face masks, a gator, or a bandana. Face covering 100 includes a face-covering portion 101 having an inner surface 102 that a wearer places against their face when in use. Face covering 100 may, also include a pair of ear loops 103.

FIGS. 2-5 illustrates various methods of providing an extract to an inner surface 102 of face covering 100. It will be appreciated by those skilled in the art that the extracts described herein may include, but are not limited to, plant extracts of cannabis or hemp, which may include THC or CBD, tobacco extracts, aromatherapy oils, as well as pharmaceuticals.

FIG. 2 illustrates a first embodiment of a method of applying an extract to a face covering. In the first embodiment, a spray device 201 is used to apply aerosolized extracts contained in the spray device as an extract mist 203 on inner surface 102.

FIG. 3 illustrates a second embodiment of a method of applying an extract to a face covering. In the second embodiment, a dropper 301 is used to apply liquid extracts contained in the dropper as drops 303 on inner surface 102.

FIG. 4 illustrates a third embodiment of a method of applying an extract to a face covering. In the third embodiment, a dispenser 401 is used to apply solid extracts 403 on inner surface 102. Dispenser 401 may be, for example and without limitation, a syringe.

FIG. 5 illustrates a fourth embodiment of a method of applying an extract to a face covering. In the fourth embodiment, the face covering 100 includes a reservoir or pad 601 on inner surface 102. Pad 601 may, for example and without limitation, be a natural or synthetic material that can retain the extract for inhaling by the wearer. The fourth embodiment may be combined with any of the first through third embodiments for applying an extract to the pad 601.

FIG. 6 is a next step for any of embodiments one through four, where wearer places face covering 100 containing extract 203, 303, or 403 on their face with inner surface 102 facing their mouth and nose. The face covering then acts as an extract delivery device by allowing for the absorption and/or inhalation of the applied extracts when placed over the mouth or nose.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment. Furthermore, the particular features, structures or characteristics may be combined in any suitable manner, as would be apparent to one of ordinary skill in the art from this disclosure, in one or more embodiments.

I claim:

1. A method of delivering an extract to a wearer of a face covering, said method comprising:
providing the extract to an inner surface of the face covering; and
placing the inner face covering on the face of the wearer.

2. The method of claim 1, where said extract is a liquid.

3. The method of claim 1, where said extract is a solid.

4. The method of claim 1, where the face covering is a face mask.

5. The method of claim 1, where the face covering is a bandana,

6. The method of claim 1, where the face covering is a gator.

7. The method of claim 1, where the extract is an extract of cannabis or an extract of hemp.

8. The method of claim 1, where the extract includes THC or CBD.

9. The method of claim 1, where the extract includes tobacco extracts.

10. The method of claim 1, where the extract includes aromatherapy oils.

11. The method of claim 1, where the extract includes pharmaceuticals.

12. The method of claim 2, where said providing the extract includes applying an aerosolized extract to the face covering.

13. The method of claim 2, where said providing the extract includes applying a liquid extract to the face covering.

14. The method of claim 2, where said providing the extract includes applying a solid extract to the face covering.

15. The method of claim 2, where said providing the extract includes applying the extract with an spray bottle.

16. The method of claim 2, where said providing the extract includes applying the extract with a dropper.

17. The method of claim 2, where said providing the extract includes applying the extract with a syringe.

18. The method of claim 2, where the inner surface includes a pad or reservoir, and where said providing the extract includes providing the extract to the pad or reservoir.
